(19) United States
(12) Patent Application Publication
(10) Pub. No.: US 2023/0108084 A1
(43) Pub. Date: Apr. 6, 2023

(54) SOLID CLEANSER COMPOSITION

(71) Applicant: Yeonjeong SHIM, Seongnam-si (KR)

(72) Inventor: Yeonjeong SHIM, Seongnam-si (KR)

(21) Appl. No.: 18/058,831

(22) Filed: Nov. 25, 2022

Related U.S. Application Data
(63) Continuation of application No. PCT/KR2021/007844, filed on Jun. 22, 2021.

(30) Foreign Application Priority Data
Jun. 26, 2020 (KR) 10-2020-0078238
Oct. 7, 2020 (KR) 10-2020-0129128

(51) Int. Cl. A61K 8/9778; A61Q 19/10; A61Q 5/02; A61Q 5/12; A61K 8/02; A61K 8/73; A61K 8/46; A61K 8/34

(52) U.S. Cl. CPC A61K 8/9778; A61K 8/0216; A61K 8/345; A61K 8/466; A61K 8/732; A61Q 5/02; A61Q 5/12; A61Q 19/10; A61K 2800/30; A61K 2800/75

(57) ABSTRACT

The present invention relates to a solid cleanser composition including starch, sodium cocoyl isethionate, glycerin, and a conditioning agent as active ingredients. The solid cleanser composition and the method for preparing the same can prevent side effects of the skin, such as skin itching and hair loss, since not including sulfate-based surfactants, and preservatives. In addition, the solid cleanser which includes sodium cocoyl isethionate, glycerin and natural plant extracts is not easily softened and does not crumble easily to provide excellent usability, and provides excellent foaming and cleaning ability.

### CROSS-REFERENCE TO RELATED APPLICATION

The present application is a continuation of International Patent Application No. PCT/KR2021/007844, filed on Jun. 22, 2021, which is based upon and claims the benefit of priority to Korean Patent Application Nos. 10-2020-0078238 filed on Jun. 26, 2020 and 10-2020-0129128 filed on Oct. 7, 2020. The disclosures of the above-listed applications are hereby incorporated by reference herein in their entirety.

### BACKGROUND

### 1. Technical Field

The present invention relates to a solid cleanser composition and a method for preparing a solid cleanser, and more particularly, to a solid type cleanser composition including a natural plant-derived surfactant, which does not include sulfate-based surfactants, chemical preservatives and preservatives that cause skin itching and hair loss, thereby providing excellent foaming ability while being usable for a long period of time due to not softening easily, and preventing hair loss and relieving itching when being applied to the scalp.

### 2. Description of Related Art

The present invention is a result made by the support of ‘Global Startup Enterprise Support Program 2022’ by Gyeonggido and Gyeonggido Business & Science Accelerator.

The present invention relates to a solid cleanser composition and a method for preparing a solid cleanser.

Cleansers are generally sold in a liquid state, are mostly comprised of water, and according to circumstances, may be up to 80% by weight of water. The liquid cleansers are not only inconvenient to carry in a case of traveling, but also cause a risk to the environment through being manufactured in an environmentally unsustainable manner. For example, the production of liquid cleansers may not be environmentally friendly due to the use of plastics for packaging, the generation of waste, the use of fresh water in a manufacturing area, and the excessive use of fuel and energy required for manufacturing and transportation.

In addition, most of the main materials of the conventional cleanser composition are chemically synthesized, and may cause skin and scalp troubles for users with sensitive skin and scalp, or cause hair loss. The use of surfactants among the raw materials of the cleanser composition is the biggest factor. Surfactants serve to improve the cleaning effect by acting on oil and fat components of the skin, hair and scalp surfaces. However, since most surfactants used conventionally are chemically synthesized and are highly toxic, they may cause skin irritation and have a negative effect on hair health.

In particular, in recent years, consumers are not satisfied with the fact that a cleanser composition has only a simple cleaning effect, and have a rising expectation for a cleanser having natural ingredients that keep skin and hair healthy, are not harmful to the human body, and are safely usable for a long period of time.

### SUMMARY

Therefore, the present inventors tried to develop a product, which is a solid type cleanser that uses materials derived from natural products with proven safety, and does not require storage containers, preservatives, or the like, and which is not easily softened, allows for convenient consumer use, and has excellent cleaning ability. As a result, the present inventors confirmed that a solid type cleanser including starch, sodium cocoyl isethionate (ISC), glycerin and natural plant extracts could be used for a long time due to not being easily softened and not crumbling easily, and had excellent foaming and cleaning efficacy. Thus, the present invention was completed.

The present invention has been made to solve the above-mentioned problems occurring in the prior art, and in an aspect of the present invention, an object of the present invention is to provide a solid cleanser composition including starch, sodium cocoyl isethionate, glycerin, and a conditioning agent as active ingredients.

Another object of the present invention is to provide a method for preparing a solid cleanser including the operations of: S1) mixing starch, sodium cocoyl isethionate, and glycerin; S2) mixing and kneading a conditioning agent in the mixture obtained above; S3) molding the dough obtained in operation S2 by hand or in a mold; and S4) drying the molded product at room temperature to obtain a solid cleanser including starch, sodium cocoyl isethionate, glycerin, and conditioning agent.

Another object of the present invention is to provide a solid cleanser prepared by the above method.

The aspects of the present invention are not limited to those mentioned above, and other aspects not mentioned herein will be clearly understood by those skilled in the art from the following description.

To accomplish the above-mentioned objects, according to an aspect of the present invention, there is provided a solid cleanser composition including starch, sodium cocoyl isethionate, glycerin, and a conditioning agent as active ingredients.

The cleanser used in the present invention may be one among shampoos, conditioners, conditioning shampoos, shower gels, liquid hand cleansers, facial cleansers, and facial body washes, and is not limited thereto, and may be cleaning products used for humans and animals.

According to an embodiment of the present invention, the starch is at least one selected from groups consisting of corn starch, tapioca starch, potato starch, wheat starch, rice starch, banana starch, and sweet potato starch. According to an embodiment of the present invention, corn starch is used, and the corn starch has excellent viscosity to strengthen adhesive force of the solid cleanser and maintain the solid form.

According to an embodiment of the present invention, the cleanser composition does not include sulfate-based surfactants and chemical preservatives.

Sodium lauryl sulfate (SLS), sodium laureth sulfate (SLES), ammonium lauryl sulfate (ALS), and ammonium laureth sulfate (ALES), which are sulfate-based surfactants, have been widely used as a cleanser for cleaning and foaming purposes. However, in a case in which such a sulfate-based surfactant remains on the skin or scalp, it may become an irritant and may be the main cause of sensitization of the skin and scalp. Sodium lauryl sulfate causes dermatitis, and sodium laureth sulfate is also known as a synthetic surfactant made by adding ethylene oxide, a primary carcinogen, to sodium lauryl sulfate.

Therefore, the present inventors looked for materials to replace the conventional sulfate-based surfactant, and found that sodium cocoyl isethionate derived from nature has low skin irritation due to skin penetration and residual performance on the skin lower than those of the synthetic surfactants, and has excellent cleaning and foaming ability. So, the present inventors applied the sodium cocoyl isethionate derived from nature to the present invention.

According to an embodiment of the present invention, with respect to the total weight of the solid cleanser composition, the solid cleanser composition including 1 to 20% by weight of starch, 40 to 80% by weight of sodium cocoyl isethionate, 1 to 20% by weight of glycerin, and 1 to 50% by weight of the conditioning agent.

With respect to the total weight of the solid cleanser composition, in a case in which sodium cocoyl isethionate is less than 40% by weight, foaming and cleaning ability of the cleanser becomes significantly lower, and in a case in which sodium cocoyl isethionate exceeds 80% by weight, it may be difficult to maintain the solid form of the cleanser and the solid cleanser may be friable. In a case in which starch exceeds 20% by weight, the cleanser may be easily softened when in contact with water, and in a case in which starch is less than 1% by weight, the solid cleanser may be friable. In a case in which glycerin exceeds 20% by weight, it may be difficult to maintain the solid form of the cleanser, and in a case in which glycerin is less than 1% by weight, the cleanser may provide low moisturizing power. In a case in which the solid cleanser is manufactured to contain the ingredients within the above-mentioned range, it may not be friable, may not be easily softened even when in contact with water, and may provide excellent foaming and cleaning ability.

The solid cleanser according to the present invention which is not in a liquid state, requires the cleansing function of the liquid cleanser but does not require viscosity stabilizer for maintaining viscosity stability according to temperature changes and thickener for preventing liquid from excessively escaping from a storage container. Moreover, the liquid cleanser may include preservatives such as paraben and benzoic acid or synthetic surfactants having the same effect to prevent the cleanser from being easily deteriorated since having a high water content, and requires a storage container, particularly uses a plastic container which is not incinerated, due to its liquid state. However, the solid cleanser is eco-friendly since not requiring the use of plastics for packaging and not generating wastes, does not include viscosity stabilizers, thickeners, preservatives, synthetic surfactants, artificial pigments, synthetic perfumes, silicon, phthalate, or the like, and is a vegan product which includes only ingredients not having undergone animal experimentation. Therefore, the solid cleanser according to the present invention does not irritate the skin, is effective to hair health, is usable in safety without being harmful to the human body, and is biodegradable so as not to pollute water and marine environments when being discharged through including only natural ingredients.

According to an embodiment of the present invention, the conditioning agent includes natural plant extracts, and the natural plant extracts include houttuynia cordata extract, green tea extract, chrysanthemum flower extract, licorice root extract, calamus root extract, angelica extract, sophora root extract, artemisia extract, and/or rosemary leaf extract.

According to an embodiment of the present invention, based on 100 parts by weight of houttuynia cordata extract, the natural plant extract includes 100 parts by weight of houttuynia cordata extract, 120 to 180 parts by weight of green tea extract, 120 to 180 parts by weight of chrysanthemum flower extract, 120 to 180 parts by weight of licorice root extract, 70 to 130 parts by weight of calamus root extract, 70 to 130 parts by weight angelica extract, 70 to 130 parts by weight of sophora root extract, 120 to 180 parts by weight of artemisia extract, and 120 to 180 parts by weight of rosemary leaf extract (Erigeron Reducing Heat 8-complex™). In an embodiment of the present invention, based on 100 parts by weight of houttuynia cordata extract, 100 parts by weight of houttuynia cordata extract, 150 parts by weight of green tea extract, 150 parts by weight of chrysanthemum flower extract, 150 parts by weight of licorice root extract, 100 parts by weight of calamus root extract, 100 parts by weight angelica extract, 100 parts by weight of sophora root extract, 150 parts by weight of artemisia extract, and 150 parts by weight of rosemary leaf extract were used.

The natural plant extracts showed hair loss prevention effect, and improvement of skin and scalp health. In an embodiment of the present invention, the solid cleanser manufactured to include the ingredients within the above-mentioned range may not be friable, may not be easily softened even when in contact with water, and provides excellent hair loss prevention effect.

Houttuynia cordata is a ground part of houttuynia cordata thumb, and the houttuynia cordata extract includes a large amount of quercitrin ingredients. The houttuynia cordata extract may remove waste of the scalp and skin. Furthermore, the houttuynia cordata extract may promote growth of hair by helping cell proliferation. In addition, the houttuynia cordata extract may provide effects of capillary expansion and toxin removal from the skin, and may be effective for chronic scalp diseases due to having a positive effect on skin inflammation.

In a case in which the solid cleanser is applied to hair and skin, the green tea extract may provide various effects of tightening pores of the skin through tannin contained in green tea leaves, cleaning hair by flavonoids, and is effective for seborrheic dandruff, itching, and hair dryness. In addition, the green tea extract provides gloss and nutrition to the hair, prevents dandruff. Additionally, the green tea extract may remove toxins, soothe acne, moisturize the skin, and may have further various effects such as sebum control, antibacterial effects, skin convergence, pore contraction, and the like through the catechin of green tea. Moreover, the green tea extract may prevent skin aging and may make skin elastic and clean by polyphenol.

The chrysanthemum flower extract may make the eyes and the head cool, may be effective for a stifling symptom due to fever in the heart, pneumonia, bronchitis, headache, shoulder discomfort, and hypertension, may also be effective for liver function, congestion, menstrual irregularity, and skin troubles such as pimples or the like, and may be further effective for dandruff, itching, and the like.

Glycyrrhizin of the licorice root extract is a saponin compound and is contained in the root in an amount of 3~7%. Glycyrrhizin is decomposed to generate glycyrrhetic acid having an adrenocortical hormone-like action, a detoxification function, and an anti-inflammatory effect.

The calamus root extract has a scalp soothing effect, and is added for facilitating hair growth and preventing hair loss.

The angelica extract includes coumarin ingredients, such as decursin, decursinol, and the like. The angelica extract is effective for alleviation of pains, drainage, hemostasis, nourishing the blood, robustness, and the like, and especially, helps the blood circulation to make the scalp and hair root healthy.

The sophora root extract may be highly effective in astriction, anti-inflammation, calming effect, curing allergic diseases, detoxification, cell regeneration, skin calming, and skin moisturization, and scalp nourishing, and the like since including matrin, oxymatrin, sopocarpine, methyl chitizine, sopolanol, anagirine, flavonoid sugar, and the like, which are alkaloids, and having lots of vitamin C and tannin.

The artemisia extract may boost immunity, may include antioxidative ingredients, may clear the head, warm the stomach, bind the bowels, and warm the hands, feet, and body.

The rosemary leaf extract may be effective in sterilization action, antiseptic action, repellent action, and inhibition of oxidization, thereby helping preservation of foodstuffs. The rosemary leaf extract may be good for colds and neuralgia, promotes vitalization, and may prevent aging. The rosemary scent which is fresh and strong may increase brain activity and improves memory and concentration. Furthermore, the rosemary leaf extract may keep the skin glossy and improve skin elasticity.

According to another embodiment of the present invention, the natural plant extract includes houttuynia cordata extract, perilla frutescens extract, rice bran extract, and oatmeal extract added to the Erigeron Reducing Heat 8-complex™ ( Erigeron Houttuynia cordata Green™). It may provide skin soothing and calming effects.

According to another embodiment of the present invention, the natural plant extract includes houttuynia cordata extract, oatmeal, and pink clay added to the Erigeron Reducing Heat 8-complex™ (Erigeron Pink Clay™) . It provides a soothing effect to the sensitive scalp.

According to another embodiment of the present invention, the natural plant extract includes houttuynia cordata extract, and oatmeal added to the Erigeron Reducing Heat 8-complex™ (Erigeron Snow Oatmeal™) .

According to another embodiment of the present invention, the natural plant extract includes houttuynia cordata extract, centella extract, perilla frutescens extract, rice bran extract, and bamboo charcoal powder added to the Erigeron Reducing Heat 8-complex™ (Erigeron Black™). It provides a sebum removal effect to an oily scalp.

According to another embodiment of the present invention, the natural plant extract includes houttuynia cordata extract, hempseed oil, mugwort extract, centella extract, and sodium cocoyl apple amino acid added to the Erigeron Reducing Heat 8-complex™ (Erigeron Hempseed™). It provides improvement of skin and scalp inflammations, and may have a calming effect.

According to another embodiment of the present invention, the natural plant extract may further include cannabis sativa leaf extract, cannabis sativa oil, and quillaja extract, or a combination thereof.

Cannabidiol (CBD) ingredients contained in cannabis sativa leaf extract, cannabis sativa oil may be effective in pain relief, inflammation reduction, anxiety relief, and sleep improvement. The CBD ingredient which includes little tetrahydrocannabinol (THC) (less than 0.3%) in the cannabis sativa ingredients is legally available in the United States. The cannabis sativa leaf extract is effective in skin soothing, skin care, muscle pain relief, scar removal, scalp care, alopecia prevention, hair care, and the like, and the cannabis sativa oil can be additionally added to the natural plant extract for anti-aging and skin calming. The quillaja extract can be additionally added to the natural plant extract for antibacterial and antiviral effects.

According to another embodiment of the present invention, the conditioning agent includes cypress powder, centella asiatica extract, Houttuynia cordata extract, brewer’s yeast extract, black bean extract, camellia flower extract, castor seed oil, camellia seed oil, argan kernel oil, and panthenol. They are ingredients of Erijeron ‘Scalp Scaling Shampoo Bar’ and may be applied to remove dead skin cells and provide moisture.

In the present invention, the “extraction” may be one among extraction, separation and fraction which are known in the art. The “extract” defined in the present invention is obtained by extraction from a natural material by using an appropriate solvent, and the extraction solvent is C1-C4 alcohol, such as water, methanol, ethanol, propanol, isopropanol, butanol, or the like, or a mixed solvent thereof. In addition, as the extraction method, there are various methods, such as hot water extraction, cold extraction, reflux cooling extraction, solvent extraction, steam distillation, ultrasonic extraction, elution, and compression. The extract may be obtained by further distillation and fraction.

The solid cleanser composition according to the present invention is not irritating to the skin since being subacid and having pH similar to that of the skin. In an embodiment of the present invention, the solid cleanser composition may have pH of about 2 to 10, about 4 to 8, about 5 to 7, and/or about 6. Preferably, the solid cleanser composition may have pH of 5 to 6.

According to another aspect of the present invention, there is provided a method for preparing a solid cleanser comprising the operations of: S1) mixing starch, sodium cocoyl isethionate, and glycerin; S2) mixing and kneading a conditioning agent in the mixture obtained above; S3) molding the dough obtained in operation S2 by hand or in a mold; and S4) drying the molded product at room temperature to obtain a solid cleanser including starch, sodium cocoyl isethionate, glycerin, and a conditioning agent.

According to an embodiment of the present invention, with respect to the total weight of the solid cleanser composition, the solid cleanser composition includes 1 to 20% by weight of starch, 40 to 80% by weight of sodium cocoyl isethionate, 1 to 20% by weight of glycerin, and 1 to 50% by weight of natural plant extracts.

According to an embodiment of the present invention, the starch is at least one selected from groups consisting of corn starch, tapioca starch, potato starch, wheat starch, rice starch, banana starch, and sweet potato starch.

According to an embodiment of the present invention, the cleanser composition does not include sulfate-based surfactants and chemical preservatives.

According to an embodiment of the present invention, the natural plant extract includes houttuynia cordata extract, green tea extract, chrysanthemum flower extract, licorice root extract, calamus root extract, angelica extract, sophora root extract, artemisia extract, and rosemary leaf extract (Erigeron Reducing Heat 8-complex™).

According to an embodiment of the present invention, the operation S3) of molding the dough obtained in operation S2 by hand or in a mold is performed at 25° C. to 35° C. In a case in which molding in the above temperature range is performed, the dough is well agglomerated, and the solid state of the cleanser is well maintained.

According to an embodiment of the present invention, the operation S3) of molding the dough obtained in operation S2 by hand or in a mold is performed at pressure of 0.7 to 1.2 bars. In a case in which molding in the above pressure range is performed, the dough is well agglomerated, and the solid state of the cleanser is well maintained.

According to another embodiment of the present invention, a solid cleanser prepared by the above method is provided. The solid cleanser prepared by the above method does not crumble easily, is not easily softened even when in contact with water, and has excellent hair loss prevention effect. Furthermore, in some cases, in order to improve the foaming performance of the solid cleanser, the solid cleanser may be packaged by a foam-enhancing packaging material, for example, a tea bag made of biodegradable plastic or ginseng or a meshtype packaging material.

### DETAILED DESCRIPTION

Hereinafter, the present invention will be described in detail with reference to preparation examples, embodiments and experimental examples.

However, the following preparation examples, embodiments and experimental Examples just illustrate the present invention in detail, and the contents of the present invention are not limited by the preparation examples, embodiments and experimental examples.

### Preparation Example 1: Preparation of Solid Cleanser According to Embodiments and Comparative Examples

In order to compare effects according to weight ratios between the cleanser composition of the present invention (Example 1) and the ingredients of the cleanser composition by the following Table 1 showing the composition, compositions of comparative examples 1 to 5 were prepared.

In order to prepare a solid cleanser, starch, sodium cocoyl isethionate, and glycerin were mixed, plant extracts were mixed and kneaded to the above mixture, and then, the dough obtained was molded by hand or in a mold. Thereafter, the molded object was dried at room temperature.

Based on 100 parts by weight of houttuynia cordata extract, 100 parts by weight of houttuynia cordata extract, 150 parts by weight of green tea extract, 150 parts by weight of chrysanthemum flower extract, 150 parts by weight of licorice root extract, 100 parts by weight of calamus root extract, 100 parts by weight angelica extract, 100 parts by weight of sophora root extract, 150 parts by weight of artemisia extract, and 150 parts by weight of rosemary leaf extract were used.

TABLE 1

| Ingredient | Embodiment | Comparative Example 1 | Comparative Example 2 | Comparative Example 3 | Comparative Example 4 | Comparative Example 5 |
|---|---|---|---|---|---|---|
| SCI | 50 | 39 | 81 | 50 | 50 | 50 |
| Starch | 10 | 11 | 4 | 0.5 | 10 | 10 |
| Glycerin | 10 | 10 | 5 | 10 | 10 | 10 |
| Plant extract | 30 | 50 | 10 | 39.5 | 30¹) | 30²) |

1) Based on 100 parts by weight of houttuynia cordata extract, 100 parts by weight of houttuynia cordata extract, 110 parts by weight of green tea extract, 110 parts by weight of chrysanthemum flower extract, 150 parts by weight of licorice root extract, 100 parts by weight of calamus root extract, 100 parts by weight angelica extract, 100 parts by weight of sophora root extract, 150 parts by weight of artemisia extract, and 150 parts by weight of rosemary leaf extract were used.

2) Based on 100 parts by weight of houttuynia cordata extract, 100 parts by weight of houttuynia cordata extract, 150 parts by weight of green tea extract, 150 parts by weight of chrysanthemum flower extract, 150 parts by weight of licorice root extract, 60 parts by weight of calamus root extract, 100 parts by weight angelica extract, 100 parts by weight of sophora root extract, 150 parts by weight of artemisia extract, and 150 parts by weight of rosemary leaf extract were used.

### 1) Experimental Example 1: Softness Test

The solid cleanser (embodiment) prepared by the method of the present invention and cleansers according to the comparative examples 1 to 3 were soaked in tap water, and then, changes in weight of the cleanser as time went by were measured to measure softness.

Weights (Wi) of the cleansers of the embodiment 1 and the comparative examples 1 to 3 were measured. Thereafter, pins were respectively put on the cleansers, and the cleansers were suspended by threads, and were soaked in tap water at 25° . The cleansers were taken out two hours later, and were dried at room temperature for about 15 minutes, and then, weights (Wf) of the cleansers were measured. Through a difference between the measured weights, softness was calculated as follows. The softness is indicated in Table 2.

$$\text{Softness} \text{=} \text{Wi} - \text{Wf}$$

TABLE 2

| Division | Softness |
|---|---|
| Embodiment | 7.5 |
| Comparative example 1 | 14.5 |
| Comparative example 2 | 9.5 |
| Comparative example 3 | 12.3 |

As a result, it was confirmed that the cleanser was softened easily in a case in which sodium cocoyl isethionate was less than 40% by weight (comparative example 1) or in a case in which starch was less than 1% by weight (comparative example 3), and that the cleanser crumbled easily even by weak power in a case in which sodium cocoyl isethionate exceeded 80% by weight.

### 2) Experimental Example 2: Foaming Ability Test

In order to confirm foaming ability of the solid cleanser (embodiment) prepared by the method according to the present invention and the cleansers according to comparative examples 1 to 3, a foaming ability test was performed. The solid cleanser according to the embodiment of the present invention and the cleansers according to the comparative examples 1 to 3 were inserted into a basin of 15 ℓ, and then, water ran down into the basin for a predetermined period of time. Thereafter, the volumes of generated foams were measured. The volumes are indicated in Table 2.

TABLE 3

| Time (Sec.) | Embodiment | Comparative Example 1 | Comparative Example 2 | Comparative Example 3 |
|---|---|---|---|---|
| 15 | 39 | 20 | 40 | 25 |
| 30 | 75 | 53 | 75 | 62 |
| 45 | 140 | 70 | 145 | 90 |
| 60 | 210 | 88 | 220 | 110 |
| 75 | 320 | 150 | 350 | 210 |
| 90 | 480 | 189 | 490 | 321 |

As a result, it was found that the foaming performance was significantly increased in the solid cleanser of the present invention (embodiment) prepared by including sodium cocoyl isethionate at a high proportion. For example, the maximum foam volume (480 mL) according to the embodiment of the present invention was about 250% higher than that (189 mL) of the comparative example 1 measured for the same period of time (90 seconds). Such a difference became greater as time passed.

On the other hand, even if sodium cocoyl isethionate was contained at a high ratio, it was confirmed that the foaming performance was rather lowered in a case in which starch was less than 1% (comparative example 3).

### 3) Experimental Example 3: Alleviation of Hair Loss and Alleviation of Itching

With respect to the solid cleanser (embodiment) prepared by the method according to the present invention and the cleansers according to comparative examples 4 and 5, tests on alleviation of hair loss and alleviation of itching were performed. Ten patients with scalp itching and ten patients with hair loss used the cleansers once a day. The results are illustrated in Table 4 below.

TABLE 4

| Division | | Test items | |
|---|---|---|---|
| | | Alleviation of hair loss | Alleviation of itching |
| Embodiment | Good | 9 | 9 |
| | Normal | 1 | 1 |
| | Bad | 0 | 0 |
| Comparative Example 4 | Good | 8 | 6 |
| | Normal | 2 | 3 |
| | Bad | 0 | 1 |
| Comparative Example 5 | Good | 6 | 8 |
| | Normal | 2 | 2 |
| | Bad | 2 | 0 |

As illustrated in Table 4, it was found that the solid shampoo composition including natural ingredients according to the present invention showed excellent effects in alleviation of scalp itching and alleviation of hair loss. The cleanser (comparative example 4) including green tea extract and chrysanthemum flower extract of natural plant extracts at a lower ratio was somewhat lower in alleviation of itching than the solid cleanser of the present invention. Moreover, the cleanser (comparative example 5) including calamus extract at a lower ratio was somewhat lower in alleviation of hair loss than the solid cleanser of the present invention.

### 4) Experimental Example 4: Reduction of Scalp Heat

With respect to the solid cleanser composition (embodiment) prepared by the method according to the present invention and the cleanser compositions according to comparative examples 4 and 5, a test on scalp heat reduction was performed. In detail, the compositions were treated to five people of 20 to 40 years of age for ten minutes, and then, the five people washed their heads and dried naturally for 20 minutes. Thereafter, temperature of the scalp on the forehead part was measured. The temperature of the scalp was measured using A-ONE SMART measuring device by the BOMTEC company. The results are illustrated in Table 5 below.

TABLE 5

| Division | Embodiment | Comparative Example 4 | Comparative Example 5 |
|---|---|---|---|
| Temperature reduction (Average) | 1.9 | 1.0 | 0.3 |

As a result, it was found that scalp heat of average 1.9° C. was reduced in a case in which the composition of the present invention was treated to the scalp, in comparison with the scalp heat before treatment of the composition.

### 5) Experimental Example 5: Test on Protein Denaturation

The solid cleanser (embodiment) prepared by the method of the present invention and a commercially purchased solid shampoo (comparative example 6) of the company L were applied to egg whites, which are known to have a protein structure similar to that of hair, to confirm whether protein denaturation occurred or not. The solid cleanser was inserted into the egg whites. After five minutes, in a case in which the color was turned blurry, it was measured as protein denaturation. The results are illustrated in Table 6 below.

TABLE 6

| | | |
|---|---|---|
| Division | Embodiment | Comparative Example 6 |
| Protein denaturation | No | Yes |

### 6) Experimental Example 6: Human Body Patch Test For Evaluating Safety

A human body patch test for evaluating safety of the solid cleanser (embodiment) of the present invention was performed. With respect to 33 adults, a diluted solution of 20 µL which was ten times diluted than the solid cleanser (embodiment) of the present invention was loaded on a patch, and a patch test was performed in a closed state for 24 hours. Skin reactions after 30 minutes, 24 hours, and 48 hours from removal of the patch were observed. A visual evaluation of the test region was performed according to the criteria of International Contact Dermatitis Research Group (ICDRG). Two or more test subjects received the test, and a higher rank was selected in a case in which judgement results of the two test subjects were different from each other. After the visual evaluation, an average skin reactivity at each test region was calculated by using the following equation, and as illustrated in Table 7, results of the human body patch test were judged based on the skin reactivity.

$$\begin{matrix}\text{Average} \text{skin} \text{reactivity} \text{=} \\ \frac{\text{Sum} \text{of} \text{judgement} \text{scores}}{\text{Highest} \text{judgement} \text{score} \times \text{Number} \text{of} \text{evaluations} \text{after} \text{removing} \text{patch} \times \text{Number} \text{of} \text{test} \text{subjects}} \times 5\end{matrix}$$

TABLE 7

| Skin reactivity | Judgement result |
|---|---|
| 0.00 ~ 0.75 | Non-irritation |
| 0.76 ~ 1.50 | Very weak irritation |
| 1.51 ~ 2.50 | Slight irritation |
| 2.51 ~ 4.00 | Intermediate irritation |
| 4.01 ~ 5.00 | Strong irritation |

As a result, since the skin reactivity to the solid cleanser of the present invention was 0.00, it was judged as non-irritation according to the criteria to judge the range from 0.00 to 0.75 as non-irritation.

### ADVANTAGEOUS EFFECTS

The solid cleanser composition and the method for preparing the solid cleanser composition according to the present invention can prevent side effects of the skin, such as skin itching and hair loss, since not including sulfate-based surfactants, and preservatives. In addition, the solid cleanser which includes sodium cocoyl isethionate, glycerin and natural plant extracts is not easily softened and does not crumble easily to provide excellent usability, and provides excellent foaming and cleaning ability.

1. A solid cleanser composition including starch, sodium cocoyl isethionate, glycerin, and a conditioning agent as active ingredients, containing 1 to 20% by weight of starch, 40 to 80% by weight of sodium cocoyl isethionate, 1 to 20% by weight of glycerin, and 1 to 50% by weight of the conditioning agent with respect to the total weight of the solid cleanser composition,
wherein based on 100 parts by weight of houttuynia cordata extract, the conditioning agent includes 100 parts by weight of houttuynia cordata extract, 120 to 180 parts by weight of green tea extract, 120 to 180 parts by weight of chrysanthemum flower extract, 120 to 180 parts by weight of licorice root extract, 70 to 130 parts by weight of calamus root extract, 70 to 130 parts by weight angelica extract, 70 to 130 parts by weight of sophora root extract, 120 to 180 parts by weight of artemisia extract, and 120 to 180 parts by weight of rosemary leaf extract.

2. The solid cleanser composition according to claim 1, wherein the starch is at least one selected from groups consisting of corn starch, tapioca starch, potato starch, wheat starch, rice starch, banana starch, and sweet potato starch.

3. The solid cleanser composition according to claim 1, wherein the cleanser composition does not include sulfate-based surfactants, and chemical preservatives.

4. A method for preparing a solid cleanser including 1 to 20% by weight of starch, 40 to 80% by weight of sodium cocoyl isethionate, 1 to 20% by weight of glycerin, and 1 to 50% by weight of the conditioning agent with respect to the total weight of the solid cleanser composition, the method comprising the operations of:
S1) mixing starch, sodium cocoyl isethionate, and glycerin;
S2) mixing and kneading a conditioning agent, which includes 100 parts by weight of houttuynia cordata extract, 120 to 180 parts by weight of green tea extract, 120 to 180 parts by weight of chrysanthemum flower extract, 120 to 180 parts by weight of licorice root extract, 70 to 130 parts by weight of calamus root extract, 70 to 130 parts by weight angelica extract, 70 to 130 parts by weight of sophora root extract, 120 to 180 parts by weight of artemisia extract, and 120 to 180 parts by weight of rosemary leaf extract, in the mixture obtained above;
S3) molding the dough obtained in operation S2 by hand or in a mold; and
S4) drying the molded product at room temperature.

5. The method according to claim 4, wherein the starch is at least one selected from groups consisting of corn starch, tapioca starch, potato starch, wheat starch, rice starch, banana starch, and sweet potato starch.

6. The method according to claim 4, wherein the cleanser does not include sulfate-based surfactants, and chemical preservatives.

7. The method according to claim 4, wherein the operation of molding the dough obtained in operation S2 by hand or in a mold is performed at 25 to 35° C.

8. A solid cleanser prepared by the method according to claim 4.
